(19) United States
(12) Patent Application Publication
(10) Pub. No.: US 2016/0287568 A1
(43) Pub. Date: Oct. 6, 2016

(54) COMPOSITION OF A NON-NUCLEOSIDE REVERSE TRANSCRIPTASE INHIBITOR

(71) Applicants: John M. BAUMANN, Bend, OR (US); Michael LOWINGER, Rahway, NJ (US); Aditya S. TATAVARTI, West Point, PA (US); Patrick Jules MARSAC, West Point, PA (US); Kristin J. M. PLOEGER, West Point, PA (US); Corey J. BLOOM, Bend, OR (US); Katherine Anne BROOKHART, Rohnert Park, CA (US); MERCK SHARP & DOHME CORP., Rahway, NJ (US)

(72) Inventors: Michael Lowinger, Jersey City, NJ (US); Aditya S. Tatavarti, Chalfont, PA (US); Patrick Jules Marsac, Philadelphia, PA (US); Kristin J.M. Ploeger, Perkasie, PA (US); John M. Baumann, Bend, OR (US); Corey J. Bloom, Bend, OR (US); Katherine Anne Brookhart, Rohnert Park, CA (US)

(73) Assignee: MERCK SHARP & DOHME CORP., Rahway, NJ (US)

(21) Appl. No.: 15/038,373

(22) PCT Filed: Nov. 19, 2014
(86) PCT No.: PCT/US2014/066281

§ 371 Date: May 20, 2016

Related U.S. Application Data
(60) Provisional application No. 61/907,537, filed on Nov. 22, 2013.

(51) Int. Cl. A61K 31/4439; A61K 9/16

(52) U.S. Cl. CPC A61K 31/4439; A61K 9/1682; A61K 9/1652

(57) ABSTRACT

The invention encompasses a composition comprising the reverse transcriptase (“RT”) inhibitor 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile sufficiently mixed in a concentration enhancing polymer, and processes for making the same. The composition and processes of the present invention significantly improve the bioavailability of the aforementioned RT inhibitor, while maintaining physical stability.

### BACKGROUND OF THE INVENTION

The reverse transcriptase (RT) inhibitor 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile (“Compound A” herein) and methods for making the same are illustrated in WO 2011/120133 A1, published on Oct. 6, 2011, and U.S. Pat. No. 8,486,975, granted Jul. 16, 2013, both of which are hereby incorporated by reference in their entirety.

Anhydrous Compound A is known to exist in at least three crystalline forms—Form I, Form II and Form III. Crystalline anhydrous Compound A has low solubility and suffers from poor bioavailability. The solubility of the most stable anhydrous crystalline form of Compound A is 6.3 μg/mL in water and fasted state simulated intestinal fluid. At a 100 mg dose, >37 liters of water are necessary to dissolve the compound.

There are many approaches for improving the bioavailability of poorly soluble drugs. Formulations that result in drug supersaturation and/or rapid dissolution may be utilized to facilitate oral drug absorption. Formulation approaches to cause drug supersaturation and/or rapid dissolution include, but are not limited to, nanoparticulate systems, amorphous systems, solid solutions, solid dispersions, and lipid systems. Such formulation approaches and techniques for preparing them are known in the art. For example, solid dispersions can be prepared using excipients and processes as described in reviews (e.g., A. T. M. Serajuddin, J Pharm Sci, 88:10, pp. 1058-1066 (1999)). Nanoparticulate systems based on both attrition and direct synthesis have also been described in reviews such as Wu et al (F. Kesisoglou, S. Panmai, Y. Wu, Advanced Drug Delivery Reviews, 59:7 pp 631-644 (2007)). Amorphous drugs dispersed in a polymer may be prepared by various methods such as spray drying or hot melt extrusion. The extrusion of drug/polymer blends has been described, see, eg, DE-A-12 248 29, EP-A-204 596; and P. Speiser, Pharmaceutica Acta Helv, 41 (1966), pp. 340.

Additionally, compounds in general will vary in their propensity to crystallize. Compound A is a strong crystallizer, i.e., it tends to crystallize very easily and therefore is difficult to maintain in an amorphous state. As a result, Compound A can readily and undesirably convert to a crystalline form during common processing conditions, creating a need for process conditions that will reduce the likelihood or eliminate such conversion.

The present invention is directed to a composition comprising Compound A in a concentration enhancing polymer and to drying processes, including spray-drying processes, for preparing said composition that maintains Compound A in amorphous form. The compositions and processes of the present invention significantly improve the bioavailability of Compound A, while maintaining physical stability.

### SUMMARY OF THE INVENTION

The invention encompasses a composition comprising the reverse transcriptase (“RT”) inhibitor 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile (Compound A) sufficiently mixed in a concentration enhancing polymer, and processes for making the same. The composition and processes of the present invention significantly improve the bioavailability of the aforementioned RT inhibitor, while maintaining physical stability.

### BRIEF DESCRIPTION OF THE DRAWINGS

FIG. 1 shows that when processing conditions in the spray drier yielding a glass transition temperature in excess of the storage temperature by 20° C. were utilized, crytallinity was never observed. However, when the difference was less than 20° C., crystallinity was observed 67% of the time. When the difference was less than 5° C., crystallinity was observed 100% of the time.

FIG. 2 shows the dissolution profiles of pharmaceutical compositions comprising an amorphous solid dispersion containing 20% drug load of Compound A and the concentration enhancing polymer HPMCAS and a formulation comprising micronized crystalline drug Compound A physically blended with a surfactant and other conventional pharmaceutical excipients. The dissolution study involved a USP II dissolution apparatus with a 100 RPM paddle speed. The non-sink dissolution experiment used a target concentration of 0.2 mg/mL in fasted state simulated intestinal fluid media.

FIG. 3 shows scatter plots demonstrating strong correlation between spray dried dispersion bulk density and tensile strength of neat spray dried intermediate (SDI) compacts. “Spray dried intermediate” refers to the spray dried composition of Compound A and HPMCAS prior to tableting.

FIG. 4 shows scatter plots demonstrating strong correlation between spray dried dispersion bulk density and tensile strength of compacts made from final formulations (pre-roller compacted).

FIG. 5 shows images of tablet defects for formulations of Compound A generated from spray dried dispersions, sprayed out of solvent X and possessing a bulk density >0.25 g/cc at commercially relevant compression speeds.

### DETAILED DESCRIPTION OF THE INVENTION

The invention encompasses compositions comprising the RT inhibitor 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile, and processes for making the same. The formulation and processes of the present invention significantly improve the bioavailability of the aforementioned RT inhibitor, while maintaining physical stability of the product over the shelf life.

For purposes of this Specification, the designation “Compound A” refers to the compound having the chemical name 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile and the following chemical structure.

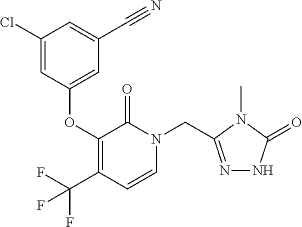 [SMILES: Cn1c(Cn2ccc(C(F)(F)F)c(Oc3cc(Cl)cc(C#N)c3)c2=O)n[nH]c1=O]

Production and the ability of Compound A to inhibit HIV reverse transcriptase is illustrated in WO 2011/120133 A1, published on Oct. 6, 2011, and U.S. Pat. No. 8,486,975, granted Jul. 16, 2013, both of which are hereby incorporated by reference in their entirety. Compound A is useful for the treatment of human immunodeficiency virus infection in humans. Compound A is known to exist in three crystalline anhydrous forms, designated as Form I, Form II and Form III, and in an amorphous form.

It is well understood that crystallization tendency varies dramatically across active pharmaceutical ingredients (Journal of Pharmaceutical Sciences, Vol. 99, No. 9, September 2010) and the rate of crystallization is a function of the thermodynamic driving force and the mobility of the system (Angell, C. A., Formation of Glasses from Liquids and Biopolymers. Science, 1995. 267(5206): p. 1924-1935. Mullin, J. W., Crystallization. 4th ed. 2001, Oxford: Reed Educational and Professional Publishing Ltd. Hoffman, J. D., Thermodynamic Driving Force in Nucleation and Growth Processes. Journal of Chemical Physics, 1958. 29(5): p. 1192-1193. Adam, G. and Gibbs, J. H., On Temperature Dependence of Cooperative Relaxation Properties in Glass-Forming Liquids. Journal of Chemical Physics, 1965. 43(1): p. 139-146. Ediger, M. D., Supercooled liquids and glasses. Journal of Physical Chemistry, 1996. 100(31): p. 13200-13212.). Compound A was found to crystallize readily in the absence of a polymer and to have a high melting point of 286° C. Neat amorphous drug generated by spray drying crystallizes within 2 weeks when stored in an open container at 5° C./ambient relative humidity (RH), 30° C./65% RH, 40° C./75% RH, and 60° C./ambient RH.

If a process exists whereby isolation of an amorphous material is possible, maintaining the amorphous material requires immobilization of the molecules. The glass transition temperature (Tg) represents a measure of mobility. In particular, molecules have high mobility above the glass transition temperature and low mobility below the glass transition temperature. As a result, if preparation of an amorphous material is possible, crystallization below the glass transition temperature is much less probable as compared to above the glass transition temperature. For pure amorphous drugs, it has been suggested that crystallization may be avoided if the temperature is maintained to 50° C. below the glass transition temperature (Hancock et al, Pharmaceutical Research, 12:6, pp 799-806 (1995)). In the presence of crystallization inhibitor, the temperature below which crystallization is avoided is not well understood. Studies which attempt to predict the crystallization tendencies of amorphous solid dispersions are prevalent in the literature—highlighting the poor level of understanding of such systems. The preparation of an amorphous solid dispersion and the conditions under which to achieve maintenance of the amorphous materials (avoidance of crystallization) is not readily ascertainable or predictable, and requires experimental assessment and engineering solutions.

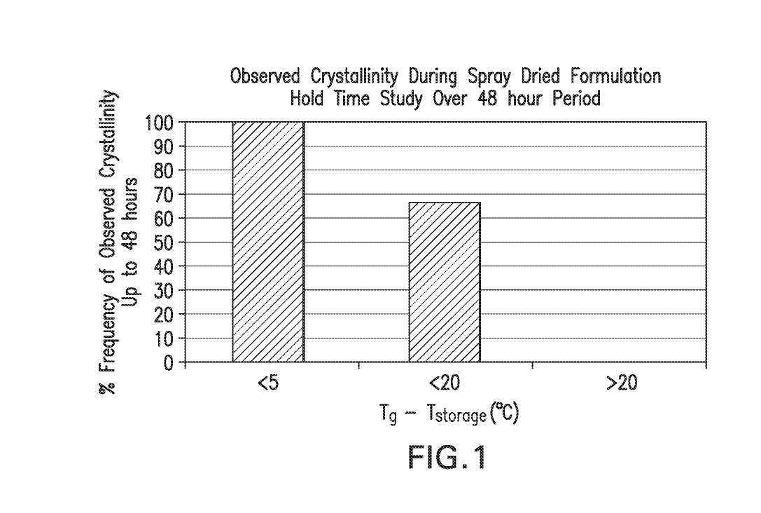

Stability studies of Compound A suggested a risk of drug crystallization over the product shelf life at relatively high drug loads as detected by x-ray powder diffraction with a limit of detection on the order of 5 wt. % on an API basis which amounts to 0.5 wt. % on a formulation basis. Crystallization would result in lower bioavailability. Moreover, the drug concentration in the formulation was further constrained in order to maintain physical stability of the product during the manufacturing process. That is, the drug product was significantly plasticized (i.e., Tg is significantly lowered) following spray drying due to residual solvent presence prior to completion of the secondary drying step. The secondary drying step is described further below. Specifically, the glass transition temperature was measured for several spray dried materials prior to secondary drying. Throughout the development process, samples were taken from the spray dryer product collection container (“collection container”), placed in a hermetically sealed differential scanning calorimetry pan, and the glass transition temperature was measured. Further, samples from the same spray drying unit operation were densely packed into vials and sealed in such a way so as to avoid loss of solvent from the bottle. The bottles were then stored at specific temperatures and monitored for crystallinity at various time points up to 48 hours. The 48-hour time frame represents a realistic production time frame from the moment the spray dried powder enters the collection container until the moment it enters the secondary drying process. Crystallization of compound A was never detected when the difference between the measured glass transition temperature and the storage temperature was greater than 20° C., i.e., when the measured Tg was greater than about 20° C. above the storage temperature. In contrast, when the difference between the measured glass transition temperature and the outlet temperature was less than 20° C., i.e., when the measured Tg was less than about 20° C. above the storage temperature, crystallization was observed about 67% of the time. Additionally, when the difference between the measured glass transition temperature and the storage temperature was less than 5° C., i.e., when the measured Tg was less than about 5° C. above the storage temperature, crystallization was observed in 100% of the samples. See FIG. 1. It was found, as illustrated in the examples, that any processing space (liquid to gas ratio, droplet size, inlet gas temperature, relative saturation, and so on) which yielded a spray dried material with sufficient residual solvent to exhibit a glass transition temperature of less than 20° C. above the storage temperature carried a substantial risk of crystallization of Compound A. This observation is in contrast with formulations of some compounds, which can be stored below the glass transition temperature of the formulation and exhibit no crystallization over a 48-hour time frame. It is also in contrast to heuristics dominant in the pharmaceutical development field suggesting that a formulation must be stored at least 50° C. above its glass transition temperature to avoid crystallization (e.g., Hancock et al, Pharmaceutical Research, 12:6, pp 799-806 (1995)). The storage temperature can be defined as the maximum temperature the spray dried powder (also referred to as particles) experiences from the moment it enters the spray dryer collection container until the moment it enters the secondary drying process. A product with minimal risk of crytallization can be produced when the difference between the measured glass transition temperature and the storage temperature exceeds 20° C., i.e., when the measured Tg is greater than about 20° C. above the storage temperature. The present invention encompasses compositions and processes that significantly improve the bioavailability of Compound A, while maintaining physical stability of the product over the shelf life.

Modification of process conditions to reduce residual solvent in the product coming out of the spray dryer in order to increase the Tg of such particles can be employed. Examples of such modifications include but are not limited to increasing drying gas temperature, reducing liquid-to-gas feed rate ratio, decreasing condenser temperature, and/or reducing droplet size. Alternatively, the storage temperature can be reduced.

In a first embodiment, the invention encompasses a composition comprising an active pharmaceutical ingredient (“API”) which is Compound A or a pharmaceutically acceptable salt thereof, sufficiently mixed in concentrating enhancing polymer and optionally one or more surfactants, wherein the composition demonstrates a measured transient concentration in excess of any of the crystalline forms of the same in any water based media. The term “sufficiently mixed” means that the resulting multi-component system lacks significant crystallinity as indicated by x-ray powder diffraction with a limit of detection on the order of 5 wt. % API basis in the final drug product. The embodiments of the invention described herein also encompass the compositions and processes wherein the API is Compound A (neutral species).

The term “concentrating enhancing polymer” means a polymer that forms an amorphous dispersion with an API, such as Compound A, that is insoluble or almost completely insoluble in water, by (a) dissolving the API or (b) interacting with the API in such a way that the API does not form crystals or crystalline domains in the polymer. A concentration-enhancing polymer is water soluble or readily dispersed in water, so that when the polymer is placed in water or an aqueous environment, also referred to herein as water based media, (e.g. fluids in the gastrointestinal (GI) tract or simulated GI fluids), the solubility and/or bioavailability of the API is increased over the solubility or bioavailability of crystalline API in the absence of the polymer.

One class of polymers suitable for use with the present invention comprises ionizable non-cellulosic polymers. Exemplary polymers include: carboxylic acid functionalized vinyl polymers, such as the carboxylic acid functionalized polymethacrylates and carboxylic acid functionalized polyacrylates, such as the EUDRAGITS copolymers, manufactured by Evonik Industries, Hanau-Wolfgang, Germany; amine-functionalized polyacrylates and polymethacrylates; proteins; and carboxylic acid functionalized starches such as starch glycolate.

Concentration enhancing polymers may also be non-cellulosic polymers that are amphiphilic, which are copolymers of a relatively hydrophilic and a relatively hydrophobic monomer. Examples include the acrylate and methacrylate copolymers (EUDRAGITS) mentioned previously. Another example of amphiphilic polymers are block copolymers of ethylene oxide (or glycol) and propylene oxide (or glycol), where the poly(propylene glycol) oligomer units are relatively hydrophobic and the poly(ethylene glycol) units are relatively hydrophilic. These polymers are often sold under the POLOXAMER® trademark.

Another class of polymers comprises ionizable and neutral cellulosic polymers with at least one ester- and/or ether-linked substituent in which the polymer has a degree of substitution of at least 0.1 for each substituent. In the nomenclature used herein, ether-linked substituents are recited prior to “cellulose” as the moiety attached to the cellulose backbone by an ether linkage; for example, “ethoxybenzoic acid cellulose” has ethoxybenzoic acid substituents on the cellulose backbone. Analogously, ester-linked substituents are recited after “cellulose” as the carboxylate; for example, “cellulose phthalate” has one carboxylic acid of each phthalate moiety ester-linked to the polymer, with the other carboxylic acid group of the phthalate group remaining as a free carboxylic acid group.

It should also be noted that a polymer name such as “cellulose acetate phthalate” (CAP) refers to any of the family of cellulosic polymers that have acetate and phthalate groups attached via ester linkages to a significant fraction of the cellulosic polymer's hydroxyl groups. Generally, the degree of substitution of each substituent group can range from 0.1 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3.

Also included within each polymer family type are cellulosic polymers that have additional substituents added in relatively small amounts that do not substantially alter the performance of the polymer. Amphiphilic cellulosics may be prepared by substituting the cellulose at any or all of the 3 hydroxyl substituents present on each saccharide repeat unit with at least one relatively hydrophobic substituent. Hydrophobic substituents may be essentially any substituent that, if substituted at a high enough level or degree of substitution, can render the cellulosic polymer essentially aqueous insoluble. Hydrophilic regions of the polymer can be either those portions that are relatively unsubstituted, since the unsubstituted hydroxyls are themselves relatively hydrophilic, or those regions that are substituted with hydrophilic substituents. Examples of hydrophobic substituents include ether-linked alkyl groups such as methyl, ethyl, propyl, butyl, etc.; or ester-linked alkyl groups such as acetate, propionate, butyrate, etc.; and ether- and/or ester-linked aryl-groups such as phenyl, benzoate, or phenylate. Hydrophilic groups include ether- or ester-linked nonionizable groups such as the hydroxyalkyl substituents hydroxyethyl, hydroxypropyl, and the alkyl ether groups such as ethoxyethoxy or methoxyethoxy. Hydrophilic substituents include those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary etherlinked non-ionizable substituents include: alkyl groups, such as methyl, ethyl, propyl, butyl, etc.; hydroxyalkyl groups such as hydroxymethyl, hydroxyethyl, hydroxypropyl, etc.; and aryl groups such as phenyl. Exemplary ester-linked non-ionizable groups include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary non-ionizable polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

In an embodiment, neutral cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

An embodiment of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid, such as ethoxynicotinic acid, and the various isomers of picolinic acid such as ethoxypicolinic acid, etc.; thiocarboxylic acids, such as 5 thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as aminosalicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer water soluble at least at pH values where any ionizable groups are ionized. In some cases, the aromatic group may itself be ionizable, such as phthalate or trimellitate substituents.

Exemplary cellulosic polymers that are at least partially ionized at physiologically relevant pH's include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Exemplary cellulosic polymers that meet the definition of amphiphilic, having hydrophilic and hydrophobic regions include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxylpropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another subset of cellulosic ionizable polymers are those that possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, and hydroxyethyl cellulose acetate succinate.

As listed above, a wide range of concentrating enhancing polymers may be used to form amorphous dispersions of Compound A in accordance with the present invention.

The compositions of the present invention may optionally comprise one or more surfactants, which may be ionic or nonionic surfactants. The surfactants can increase the rate of dissolution by facilitating wetting, thereby increasing the maximum concentration of dissolved drug. The surfactants may also make the dispersion easier to process. Surfactants may also stabilize the amorphous dispersions by inhibiting crystallization or precipitation of the drug by interacting with the dissolved drug by such mechanisms as complexation, formation of inclusion complexes, formation of micelles, and adsorption to the surface of the solid drug. Suitable surfactants include cationic, anionic, and nonionic surfactants. These include for example fatty acids and alkyl sulfonates; cationic surfactants such as benzalkonium chloride (Hyamine 1622, available from Lonza, Inc., Fairlawn, N.J.); anionic surfactants, such as dioctyl sodium sulfosuccinate (Docusate Sodium, available from Mallinckrodt Spec. Chem., St. Louis, Mo.) and sodium lauryl sulfate (sodium dodecyl sulfate); sorbitan fatty acid esters (SPAN series of surfactants); Vitamin E TPGS; polyoxyethylene sorbitan fatty acid esters (Tween series of surfactants, available from ICI Americas Inc., Wilmington, Del.); polyoxyethylene castor oils and hydrogenated castor oils such as Cremophor RH-40 and Cremopher EL; Liposorb P-20, available from Lipochem Inc., Patterson N.J.; Capmul POE-0, available from Abitec Corp., Janesville, Wis.), and natural surfactants such as sodium taurocholic acid, 1-palmitoyl-2-oleoyl-sn-glycero-3-phosphocholine, lecithin, and other phospholipids and mono- and diglycerides.

The compositions of the present invention may optionally comprise other excipients, such as one or more disintegrants, diluents or lubricants. Representative disintegrants may include croscarmellose sodium, sodium starch glycolate, crospovidone, and starch. Representative glidants may include silicon dioxide and talc. Representative lubricants may include magnesium stearate, stearic acid, and sodium stearyl fumarate. Representative diluents may include microcrystalline cellulose, lactose, and mannitol.

The compositions of the present invention are prepared by processes that are suitable for causing a compound (the drug) to form a dispersion (also referred to as an amorphous dispersion, a solid dipersion, solid solution, or amorphous solid dispersion) in the polymer such that the drug is generally amorphous. The dispersions are stable, and the drug does not form detectable crystals or other insoluble particles. Such methods include solution methods, such as spray drying, spray coating, freeze-drying, and evaporation of a co-solvent under vacuum or by heating a solution of polymer and drug. Such methods also include methods that mix the solid drug with the polymer in the molten state, such as hot melt extrusion, and methods of compounding the solid non-molten polymer and drug under heat and pressure to form a dispersion. Precipitation methods (e.g. solvent, anti-solvent) may also be utilized.

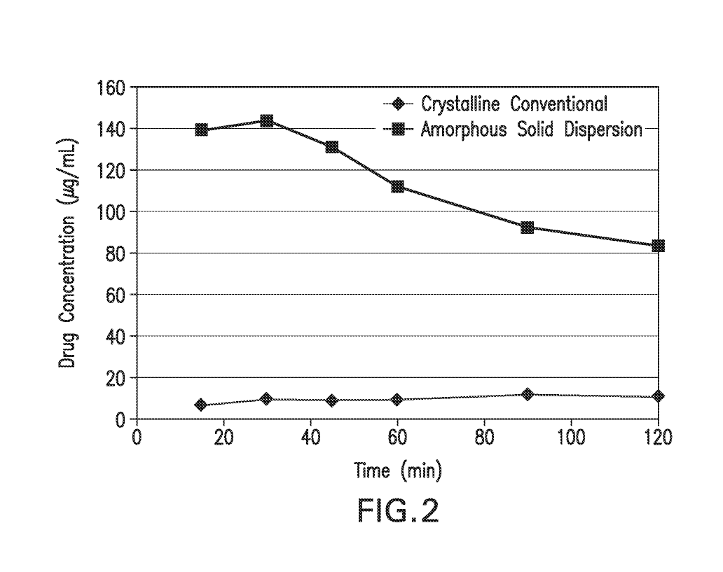

The compositions comprising the concentration-enhancing polymer increase the concentration of Compound A in an aqueous environment, such as water, the gastrointestinal (GI) tract, or a simulated GI fluid prepared for in vitro laboratory tests relative to a control composition comprising an equivalent amount of crystalline Compound A without polymer. Once the composition is introduced into an aqueous environment, the composition comprising the concentration-enhancing polymer and Compound A provides a higher maximum aqueous concentration of Compound A relative to a control composition having the same concentration of Compound A but without the concentration-enhancing polymer. An inert filler may be used in place of the polymer in the control to keep the Compound A at the same concentration as in the composition comprising the polymer. See FIG. 2.

As shown in the examples that follow, in vivo pharmacokinetics measurements in which the concentration of Compound A is measured as a function of time in blood or serum after administration of the formulation to a test animal, the compositions of the present invention exhibit an area under the concentration versus time curve (AUC) and a maximum concentration Cₘₐₓ that is greater than that of a control composition comprising an equivalent quantity of the compound without the concentration-enhancing polymer. The compositions disclosed herein exhibit improved in vivo bioavailability compared with formulations that do not have the concentration-enhancing polymer. The AUC of the drug and the maximal concentration of the drug in the blood or serum are increased when the formulations are administered to a patient.

The compositions of Compound A and concentration-enhancing polymer is prepared according to processes which results in at least a major portion of Compound A present in the amorphous state relative to other morphological forms of Compound A, at least preferably 95%. These processes include mechanical processes, such as milling and extrusion; melt processes, such as high temperature fusion, hot melt extrusion, solvent modified fusion, and melt congealing processes; and solvent processes, including non-solvent precipitation processes, spray coating, and spray-drying. Although the dispersions of the present invention may be made by any of these processes, in an embodiment of the invention Compound A in the pharmaceutical composition is substantially amorphous and is substantially homogeneously distributed throughout the polymer. The relative amounts of crystalline and amorphous Compound A can be determined by several analytical methods, including for example, differential scanning calorimetry (DSC), x-ray powder diffraction (XRPD) and Raman spectroscopy.

In an embodiment of the invention, processes for making compositions of Compound A with a concentration-enhancing polymer include (a) hot melt extrusion and (b) spray drying. In a further embodiment of the present invention, polymers for use in these processes are polyvinylpyrrolidinone, polyvinylpyrrolidinone-polyvinylacetate copolymers (for example copovidone), HPC, HPMCAS, HPMC, HPMCP, CAP, and CAT. In an embodiment of the present invention, polymers for use in hot melt extrusion are polyvinylpyrrolidinone and polyvinylpyrrolidinone-polyvinylacetate copolymers (copovidone such as Kollidon VA64 or Plasdone S630). In an embodiment of the present invention, polymers for spray drying include HPC, HPMCAS, HPMC, HPMCP, CAP, and CAT. In an embodiment of the present invention, the polymer for spray drying is HPMCAS.

Techniques for spray drying and hot melt extrusion are known in the art. In spray drying, the polymer, active compound, and other optional ingredients, such as surfactants, are dissolved in a solvent, or mixture of solvents, and are then sprayed through a nozzle or atomiser as a fine spray into a spray drying chamber where the solvent is evaporated quickly to make fine particles comprising polymer, drug, and optional other ingredients. The solvent is any solvent in which all of the components of the composition are soluble. The solvent should also be suitable for use in preparing pharmaceutical compositions. Exemplary solvents are acetone, methanol, ethanol and tetrahydrofuran. In hot melt extrusion, the polymer, drug, and optional surfactants are mixed together, and then the mixture of polymer, drug and surfactant are fed into the chamber of an extruder, preferably a twin screw extruder to obtain better mixing, and are then thoroughly melted and mixed to make an amorphous dispersion.

In an embodiment, the invention encompasses a pharmaceutical composition comprising an effective amount of an active pharmaceutical ingredient which is 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile or a pharmaceutically acceptable salt thereof and a concentrating enhancing polymer and optionally one or more surfactants, wherein the concentrating enhancing polymer is selected from the group consisting of: hydroxypropyl methyl cellulose acetate succinate, hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone, or polyvinylpyrrolidinone-polyvinylacetate copolymers, wherein the active pharmaceutical ingredient is in substantially amorphous form dispersed in the concentrating enhancing polymer.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV reverse transcriptase (wild type and/or mutant strains thereof) and thereby elicit the response being sought (i.e., an “inhibition effective amount”). When the active compound (i.e., active ingredient) is administered as the salt, references to the amount of active ingredient are to weight of the free form (i.e., the non-salt form) of the compound.

The term “substantially amorphous form” means that the active pharmaceutical ingredient dispersed in the concentrating enhancing polymer lacks significant crystallinity as indicated by x-ray powder diffraction with a limit of detection on the order of 5 wt. % API basis in the final drug product.

In an embodiment, the invention encompasses a pharmaceutical composition comprising an API which is Compound A or a pharmaceutically acceptable salt thereof sufficiently mixed in a concentrating enhancing polymer and optionally one or more surfactants, wherein the concentrating enhancing polymer is selected from the group consisting of: hydroxypropyl methyl cellulose acetate succinate (HPMCAS), hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate, cellulose acetate isophthalate, polyvinylpyrrolidinone, and polyvinylpyrrolidinone-polyvinylacetate copolymers. In an embodiment, the polymer is hydroxypropyl methyl cellulose acetate succinate (HPMCAS).

In another embodiment, the invention encompasses a pharmaceutical composition comprising an API which is Compound A or a pharmaceutically acceptable salt thereof sufficiently mixed in a concentrating enhancing polymer and optionally one or more surfactants, wherein the concentrating enhancing polymer is hydroxypropyl methyl cellulose acetate succinate (HPMCAS), wherein the drug load of the active pharmaceutical ingredient is from about 5% to about 40%.

In another embodiment, the invention encompasses a pharmaceutical composition comprising an API which is Compound A or a pharmaceutically acceptable salt thereof sufficiently mixed in a concentrating enhancing polymer and optionally one or more surfactants, wherein the concentrating enhancing polymer is hydroxypropyl methyl cellulose acetate succinate (HPMCAS), wherein the drug load of the active pharmaceutical ingredient is from about 10% to about 40%.

In another embodiment, the invention encompasses a pharmaceutical composition comprising an API which is Compound A or a pharmaceutically acceptable salt thereof sufficiently mixed in a concentrating enhancing polymer and optionally one or more surfactants, wherein the concentrating enhancing polymer is hydroxypropyl methyl cellulose acetate succinate (HPMCAS), wherein the drug load of the active pharmaceutical ingredient is from about 10% to about 30%.

In another embodiment, the invention encompasses a pharmaceutical composition comprising an API which is Compound A or a pharmaceutically acceptable salt thereof sufficiently mixed in a concentrating enhancing polymer and optionally one or more surfactants, wherein the concentrating enhancing polymer is hydroxypropyl methyl cellulose acetate succinate (HPMCAS), wherein the drug load of the active pharmaceutical ingredient is from about 15% to about 25%.

In the embodiments described above, said composition may comprise one or more surfactants selected from the group consisting of anionic surfactants and nonionic surfactants (“sixth embodiment”). In a further embodiment, the one or more surfactants is selected from sodium dodecyl sulfate and one or more nonionic surfactants selected from (a) sorbitan fatty acid esters, (b) polyoxyethylene sorbitan fatty acid esters, (c) polyoxyethylene castor oils, (d) polyoxyethylene hydrogenated castor oils, and (e) vitamin E TPGS; and mixtures thereof.

For purposes of this specification, the term “drug load” means the level of API, on a weight basis, in the composition coming out of the primary drying process (e.g., spray drying). The drug loads of the spray dried intermediates shown in Table 1 are illustrative.

In an embodiment, the invention encompasses a process for making a composition comprising an API which is Compound A or a pharmaceutically acceptable salt thereof sufficiently mixed in a concentrating enhancing polymer, comprising
(a) dissolving 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile and the concentrating enhancing polymer in a solvent system that results in a stable, single-phase dispersion; and
(b) drying the solution from step (a).
   Drying the solution in step (b) can be accomplished using known techniques in the art, for example spray-drying, freeze drying, rotavaping (rotary evaporation), radiation-assisted drying, humid drying, and film evaporation.

In another embodiment, the invention encompasses the process according to the aforementioned embodiment wherein the solvent system is acetone/water.

In another embodiment, the invention encompasses the process according to the aforementioned embodiments wherein the drying in step (b) is achieved using a spray drying process comprising:

(i) delivering the solution from step (a) to an atomiser of a spray-drying apparatus;

(ii) dispersing the solution from step (a) into droplets by passing the solution through the atomiser into a drying chamber of the spray-drying apparatus;

(iii) mixing the droplets in the drying chamber with a drying gas (e.g., an inert gas, air, or particularly N₂,) which flows at a drying gas flow rate through the drying chamber from an inlet to an outlet of the drying chamber, whereby solvent from the solvent system is evaporated to make particles comprising the concentrating enhancing polymer and active pharmaceutical ingredient, and

(iii) separating the particles from the drying gas and collecting the particles.

Spray-drying apparatuses and atomisers that can be used in the present invention are known in the art. Examples of atomisers include two-fluid nozzles, piezoelectric nozzles, ultrasonic nozzles, and pressure-swirl nozzles. Techniques for spray-drying are described in the literature and well known in the art, and further exemplified in the examples that follow. Independent spray drying process parameters, such as atomizer type, liquid flow rate, drying gas flow rate, atomization gas flow rate, inlet and outlet temperatures, have complex interactive impacts on the process that can be determined through a combination of modeling and experimentation. The particles can be separated and collected in the spray-drying apparatus by using for example a cyclone. Raw material inputs to the process may also substantially impact quality, including solvent system. The quality of the process can be determined by its ability to avoid crystallization of the active pharmaceutical ingredient in the solid-state and to enable the active pharmaceutical ingredient to remain in solution above its crystalline equilibrium solubility for a physiologically relevant length of time.

Another embodiment of the invention encompasses the process according to any of the aforementioned embodiments wherein the composition is a pharmaceutical composition and the active pharmaceutical ingredient is present in an effective amount.

Another embodiment of the invention encompasses the process according to any of the aforementioned embodiments wherein the particles comprising the concentrating enhancing polymer and active pharmaceutical ingredient resulting from step (b) is subsequently subjected to a secondary drying process comprising mixing the particles with a second drying gas to evaporate residual solvent from the solvent system, wherein the temperature and gas flow rate of the drying gas are such to ensure that the active pharmaceutical ingredient remains <5% crystalline from the moment the particles are collected until the completion of the process. Secondary spray drying processes are known in the art and include for example tray drying, tumble drying, fluid-bed drying, contact drying, and vacuum drying. In an embodiment, the drying conditions of the secondary drying process are selected to maintain the humidity during the secondary drying process below the glass transition temperature (Tg) of the particles comprising the concentrating enhancing polymer and active pharmaceutical ingredient resulting from step (b). In another embodiment, the humidity is less than about 15% RH.

In another embodiment, the invention encompasses the spray drying process of step (b) wherein the spray drying process produces spray dried particles comprising the concentrating enhancing polymer and active pharmaceutical ingredient having a glass transition temperature that is about 5° C. or more above the storage temperature of the spray dried particles. The storage temperature is the maximum temperature the spray dried particles experience from the moment the particles enter the spray dryer collection container until the start of the secondary drying process.

In another embodiment, the invention encompasses the spray drying process of step (b) wherein the spray drying process produces spray dried particles comprising the concentrating enhancing polymer and active pharmaceutical ingredient having a glass transition temperature that is greater than about 10° C. above the storage temperature of the spray dried particles.

In another embodiment, the invention encompasses the spray drying process of step (b) wherein the spray drying process produces spray dried particles comprising the concentrating enhancing polymer and active pharmaceutical ingredient having a glass transition temperature that is about 20° C. or more above the storage temperature of the spray dried particles.

In another embodiment, the invention encompasses the spray drying process of step (b) wherein the spray drying process produces spray dried particles comprising the concentrating enhancing polymer and active pharmaceutical ingredient having a glass transition temperature that is greater than about 20° C. above the storage temperature of the spray dried particles.

In another embodiment, the invention encompasses above described spray drying process using a temperature of the drying gas at the inlet of the drying chamber and ratio of spray solution flow rate to drying gas flow rate to ensure the glass transition temperature of particles comprising the concentrating enhancing polymer and active pharmaceutical ingredient is greater than about 5° C. over the temperature of the spray dried powder from the moment the particles are collected until the start of the secondary drying process.

In another embodiment, the invention encompasses above the described spray drying process using a solvent system to ensure the glass transition temperature of the particles comprising the concentrating enhancing polymer and active pharmaceutical ingredient is greater than about 10° C. over the temperature of the spray dried powder from the moment the particles are collected until the start of the secondary drying process.

In another embodiment, the invention encompasses above the described spray drying process using a solvent system to ensure the glass transition temperature of the particles comprising the concentrating enhancing polymer and active pharmaceutical ingredient is greater than about 20° C. over the temperature of the spray dried powder from the moment the particles are collected until the start of the secondary drying process.

In another embodiment, the invention encompasses the above-described spray drying process wherein prior to delivering the solution from step (a) to the atomiser of a spray-drying apparatus, the solution from step (a) is contacted with a heat exchanger to deliver the solution at an elevated temperature. The term “elevated temperature” means a temperature above ambient temperature but below the boiling point of the solvent system. Such techniques are illustrated, for example in WO 2010/111132, published on Sep. 30, 2010.

Another embodiment encompasses the process of the aforementioned embodiment wherein the pharmaceutical composition is in the form of a tablet, further comprising blending the dispersion particles, comprising the concentrating enhancing polymer and active pharmaceutical ingredient following the secondary drying process, with one or more diluents, and optionally one or more previously described functional excipients such as disintegrants, glidants, lubricatns and other excipients, to form a mixture, granulating the mixture, followed by compression of the granulated mixture to form the tablet.

The formulations of the present invention may also be in other dosage forms, such as capsules, oral granules, powder for reconstitution, lyophilization cake, soft chews, orally dissolving films, and suspensions.

Another embodiment encompasses the process of the aforementioned embodiment for compound A wherein the dispersion particles, comprising the concentrating enhancing polymer and active pharmaceutical ingredient, used in the tablet formulation, possess a bulk density in the range of 0.1-0.3 g/cc.

Another embodiment encompasses a process for making a pharmaceutical composition comprising an active pharmaceutical ingredient which is 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile or a pharmaceutically acceptable salt thereof sufficiently mixed in a concentrating enhancing polymer, said process comprising dissolving the active pharmaceutical ingredient and the concentrating enhancing polymer in a solvent system and subsequently creating a supersaturated condition so as to precipitate a solid from said solution. In another embodiment, the invention encompasses said process further comprising dissolving the active pharmaceutical ingredient and the concentration enhancing polymer in a solvent with subsequent addition of an anti-solvent, or changing the temperature, so as to precipitate the active pharmaceutical composition and concentrating enhancing polymer from the solution.

The invention also encompasses a pharmaceutical composition comprising an active pharmaceutical ingredient which is 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile or a pharmaceutically acceptable salt thereof sufficiently mixed in a concentrating enhancing polymer, wherein the pharmaceutical composition is made by any of the aforementioned processes described above.

### Examples

Examples of preparations of spray dried pharmaceutical formulations of Compound A are provided below. A goal of developing a solid dispersion formulation is to enable superior bioperformance relative to a conventional formulation containing crystalline Compound A. Biopharmaceutical comparisons for the spray dried solid dispersion formulation containing the concentration enhancing polymer are made with conventional formulations containing the same amount of the API. The API is Compound A, or a pharmaceutically acceptable salt thereof. Bioavailability is determined in vivo by dosing trial formulations and/or other formulations of the active pharmaceutical ingredient (API) to Beagle dogs at a dose of 1 mg/kg of the API and then measuring the amount of API in the serum or blood as a function of time.

### Preparation of Spray Dry Formulations

Spray dried formulations comprised of Compound A (5-30% w/w); an optional surfactant (1-10% w/w) such as SDS (sodium dodecyl sulfate), Vitamin E TPGS, Polysorbate 80, Span 80, or Cremophor EL, or a mixture of two or more of these surfactants; and a concentration enhancing polymer such as HPMCAS-L, HPMCAS-M, or HPMCAS-H. The components were dissolved or suspended in a solvent system, such as acetone, methanol, tetrahydrofuran and mixtures of organic solvents with water (0.5-7% w/w solids), and then spray dried as described below.

### Solution Preparation & Spray Drying Process I:

Compound A, optional surfactant or surfactants, and polymer were mixed with acetone, methanol, tetrahydrofuran (THF) or mixtures of organic solvents with water using a mechanical agitator, yielding a solution/structured suspension wherein all the API is in solution and a portion of the polymer may exist as a colloidal suspension. The API and the optional surfactant are added first to ensure complete dissolution as confirmed by a clear solution. Following this, the HPMCAS is added and the contents stirred over 1-2 hours to facilitate polymer dissolution.

Spray drying was carried out in a NIRO SD Micro spray drier. Nitrogen gas and the spray solution were fed concurrently into a two-fluid nozzle and sprayed into the drying chamber, along with additional heated nitrogen, resulting in rapid evaporation of the droplets to form solid dispersion particles. The dried dispersion particles are conveyed by the processing gas into a cyclone and then into a bag filter chamber for collection. The solution feed rate was controlled by an external peristaltic pump, and is ˜5-20 gm/min on a laboratory scale. The atomizing nitrogen rate and processing nitrogen rates were 2-3 kg/hr for atomizing nitrogen and 20-30 kg/hr for processing nitrogen. The targeted processing gas temperature at the drying chamber outlet was slightly below the boiling point of the solvent system and the inlet chamber temperature (at the outlet of the nozzle) was adjusted to obtain the desired outlet temperature. An inlet temperature set point of 110-120° C. was typical.

### Solution Preparation & Spray Drying Process II:

The solution preparation process is similar to that described for Process 1. Spray drying was carried out in a NIRO PSD-1 extended chamber spray drier equipped with a pressure-swirl nozzle. The solution flow rate was in the 150-250 g/min range with the outlet temperature in the 35-60° C. range and the inlet temperature in the 120-160° C. range. The feed pressure was in the 80-400 psig range with the processing gas flow rate in the 1500-2000 g/min range. The process can be run in either a single pass or a recycle mode. When employing the recycle mode, the condenser temperature is set at −10° C.

### Solution Preparation & Spray Drying Process III:

A third approach is to add the polymer (HPMCAS) to the solvent and mix for 1-3 hours. Then add Compound A at 1.4% w/w concentration, thereby making an opaque solution. Then, increase the spray solution temperature above ambient conditions but below the boiling point of the solvent at atmospheric pressure, so as to ensure Compound A is in solution. The solution lines connecting the solution tank and the spray dryer are insulated to prevent thermal loss. An in-line heat exchanger prior to the spray nozzle reheats the solution back to 50° C. This approach increases the overall solids concentration in the solution.

Spray drying was carried out in a NIRO PSD-2 spray drier equipped with a pressure-swirl nozzle. The solution flow rate was in the 35-40 kg/hr range with the outlet temperature in the 40-55° C. range and the inlet temperature in the 110-140° C. range. The feed pressure was in the 400-500 psig range with the processing gas flow rate at ˜400 kg/hr. The condenser temperature is set at −10° C.

### Post Spray Drying Processing:

The spray dried material is collected from the cyclone area and secondary dried. The secondary drying is carried out in either a tray dryer or a contact dryer. The solvent content in the spray dried material following secondary drying is generally in the 0.1-0.5% w/w range. The particle size and the bulk density of the spray dried particles are key physical parameters evaluated. The process is designed such that the D(50) of the material is in the 15-30 μm range with the D(90) in the 50-70 μm range and the bulk density is in the 0.22-0.29 g/cc range.

The spray drying process is not often immediately followed by the secondary drying process for the removal of residual solvents. There is generally a hold time for logistical reasons between the two steps which is herein referred to as the “wet hold time”. Due to the high residual solvent in the spray dried material and consequently lower glass transition temperature, the wet hold time period is a key risk for drug crystallization. Based on the length of this hold time and the conditions that the batch is exposed to, the extent of crystallization can vary.

### Drug Load

Table 1 shows the physical stability of spray dried composition of Compound A and HPMCAS after secondary drying of different drug load prepared under conditions whereby crystallization was avoided during the manufacturing process. Each batch was placed at the storage condition 40° C. and 75% RH and monitored for drug crystallization over time using x-ray powder diffraction (XRPD). Increasing the drug load will reduce the Tg. The data below further supports the proposition that reducing the Tg at a given storage temperature puts the composition at increased risk for crystallization.

TABLE 1
Recrystallization from SDI Observed by XRPD

40 C./75% RH open

| DL | Initial | 4-week | 8-week | 16-week | 26-weeks |
|---|---|---|---|---|---|
| 20% | No | No | No | No | No |
| 25% | No | No | No | No | No |
| 30% | No | No | No | No | No |
| 35% | No | No | No | Yes | N/T |
| 40% | No | No | Yes | Yes | N/T |

N/T = not tested

As shown in Table 1, drug formulations of the invention having drug loads of 20%, 25% and 30% surprisingly showed no recrystallization over a 26 week period. However, crystallization was observed at 35% drug loading after 16 weeks of storage and at 40% drug loading after 8 weeks of storage.

### Process Conditions

Crystallization can be detected by techniques such as X-ray powder diffraction, differential scanning calorimetry, scanning electron microscopy (SEM), or another suitable technique. SEM was used to understand the crystallization potential of a certain formulation of Compound A in relation to the process conditions used. The results are summarized in Table 2 and demonstrate that the probability of achieving a stable spray dried intermediate is a strong function of the difference between the storage temperature (Tstorage) and the glass transition temperature measured in the presence of residual spray dry solvent (Tgwet). Based on these data, it is implied that a composition having substantially amorphous Compound A is produced under processing conditions which provide a Tgwet−Tstorage of greater than 20° C. No crystallization was observed when Tgwet−Tstorage>20° C., irrespective of solvent system, drug load and any other process conditions. Table 2 shows examples of successful spray dry manufactures. These results are also represented in FIG. 1.

TABLE 2

| | | Storage | | | Crystal- |
|---|---|---|---|---|---|
| Drug | | Temper- | | | lization |
| loading | | ature | | Tgwet − | observed at |
| (% | Solvent | (Tstorage, | Tgwet | Tstorage | 48 hours? |
| w/w) | system | ° C.) | (° C.) | (° C.) | (Y/N) |
| 25% | THF:Water | 52.0 | 48.5 | −3.5 | Yes |
| 25% | THF/Water | 44.0 | 44.5 | 0.5 | Yes |
| 20% | Acetone:Water | 58.0 | 61.9 | 3.9 | Yes |
| 20% | Acetone/Water | 46.0 | 54.6 | 8.6 | Yes |
| 25% | Acetone/Water | 47.0 | 58.2 | 11.2 | No |
| 20% | Acetone/Water | 47.0 | 58.9 | 11.9 | Yes |
| 25% | Acetone/Water | 40.0 | 54.6 | 14.6 | No |
| 25% | Acetone/Water | 39.0 | 57.2 | 18.2 | Yes |
| 20% | Acetone/Water | 54.0 | 75.2 | 21.2 | No |
| 20% | Acetone/Water | 47.0 | 69.1 | 22.1 | No |
| 20% | Acetone/Water | 54.0 | 77.4 | 23.4 | No |
| 20% | Acetone/Water | 40.0 | 63.8 | 23.8 | No |
| 20% | Acetone/Water | 54.0 | 78.0 | 24.0 | No |
| 20% | Acetone/Water | 47.0 | 72.1 | 25.1 | No |
| 20% | Acetone/Water | 54.0 | 80.5 | 26.5 | No |
| 25% | THF/Water | 40.0 | 66.6 | 26.6 | No |
| 20% | Acetone/Water | 54.0 | 80.6 | 26.6 | No |
| 20% | Acetone/Water | 40.0 | 70.8 | 30.8 | No |
| 20% | Acetone/Water | 40.0 | 76.1 | 36.1 | No |
| 20% | Acetone/Water | 40.0 | 77.8 | 37.8 | No |
| 20% | Acetone/Water | 40.0 | 80.9 | 40.9 | No |

### Pharmaceutical Formulation

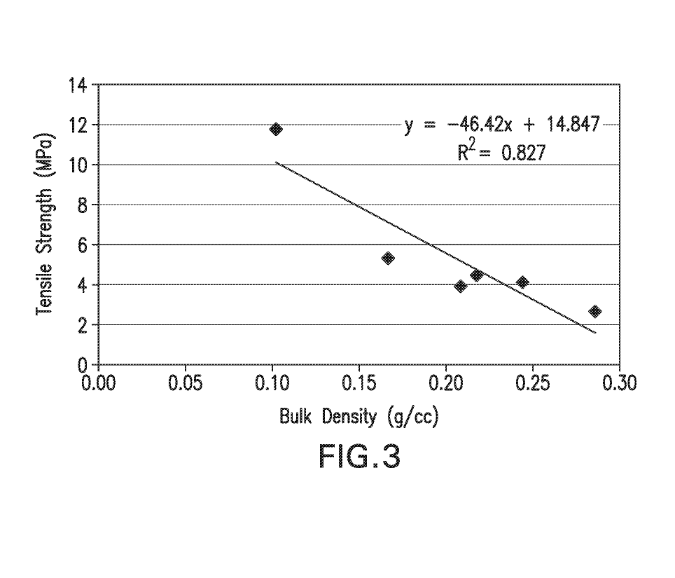

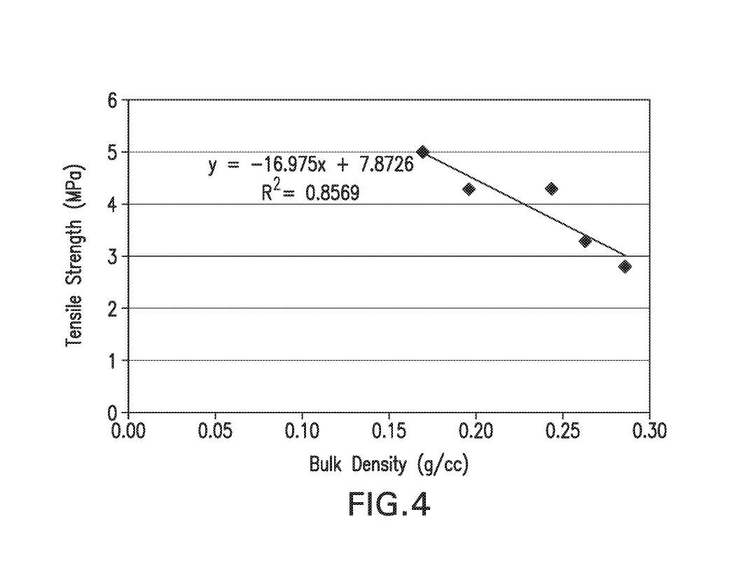

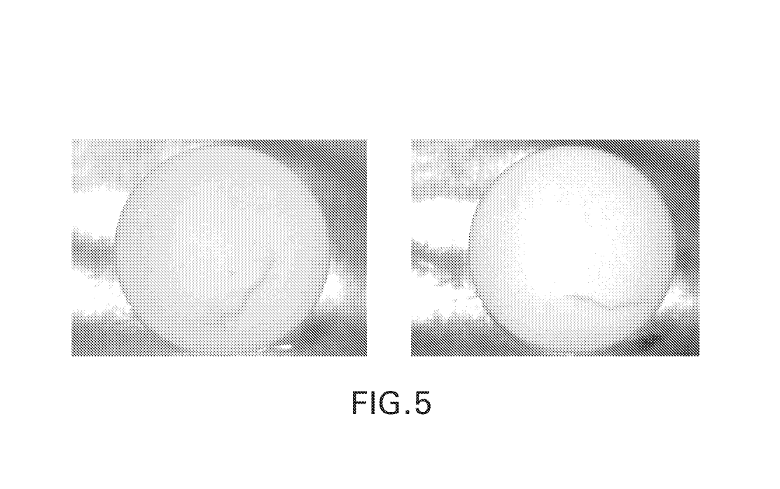

A final pharmaceutical formulation is comprised of spray dried dispersion with specific particle attributes, combined with tableting excipients and processed under controlled conditions (both spray drying and downstream) that favor formation of a tablet with desired tensile strength. The compactability of the formulations is intricately linked to the spray solvent and the resulting bulk density of spray dried dispersion. For a specific solvent system, it has been found that there is a strong correlation between the bulk density of the spray dried dispersion and tensile strength of compacts of neat spray dried intermediate (SDI) and formulations thereof. See FIG. 3 and FIG. 4. Consequently, application of a favorable solvent system and process conditions in accordance with the invention are employed to optimize the product from a mechanical integrity standpoint. In certain cases, tablets of formulations containing spray dried intermediates with high bulk density show failure upon compression, due to low tensile strength. FIG. 5 depicts images of tablet defects for unoptimized formulations generated from spray dried dispersions, sprayed out of acetone/water and possessing a bulk density >0.25 g/cc at commercially relevant compression speeds.

Table 3 illustrates a formulation in accordance of the invention.

TABLE 3

| | | Composition |
|---|---|---|
| Components | Function | (% w/w) |
| Compound A:Hydroxypropyl | Spray Dried | 50.0 |
| methylcellulose acetate | Dispersion | |
| succinate (HPMCAS-LG) | | |
| (20:80) | | |
| Lactose monohydrate, (Fast Flo 316) | Diluent/ | 21.5 |
| | Tableting Aid | |
| Microcrystalline cellulose (Avicel | Diluent/ | 21.5 |
| PH102) | Tableting aid | |
| Silicon Dioxide (Intragranular) | Glidant | 0.50 |
| Croscarmellose Na (Intragranular) | Disintegrant | 3.00 |
| Magnesium stearate (Intragranular) | Lubricant | 0.25 |
| Croscarmellose Na (Extragranular) | Disintegrant | 3.00 |
| Magnesium stearate (Extragranular) | Lubricant | 0.25 |
| Total | N/A | 100 |

### Biopharmaceutical Evaluation

The spray dried particles from Spray Dry Process I were made into granules as follows. The particles were blended in a suitable blender (V or Bohle) with microcrystalline cellulose (diluent/compression aid), lactose (diluent/compression aid), croscarmellose sodium (a disintegrant), colloidal silicon dioxide (a glidant), and magnesium stearate (a lubricant). The blended powders were then roller compacted into granules, subjected to extragranular lubrication, and filled into capsules.

A formulation prepared as described above that comprised of 10% (w/w) Compound A, 40% HPMCAS-LG, 22.75% lactose monohydrate, 22.75% microcrystalline cellulose, 3% croscarmellose sodium, 0.5% colloidal silicon dioxide, and 1% magnesium stearate was transferred to capsules, with each capsule containing 10 mg of Compound A. The pharmacokinetic profile of this composition was tested in a panel of 3 fasted beagle dogs with a dose of 1 mg/kg. The pharmacokinetic measurements of Compound A in the blood for a period of 24 hours was as follows: AUC₀₋₂₄ is 137±25.3 μM*hr; Cₘₐₓ is 7.23±0.99 μM; and Tₘₐₓ is 4.0 hr.

For comparison, a formulation containing Compound A without the concentration enhancing polymer was made by blending and encapsulating 30% of crystalline Compound A, 12.2% microcrystalline cellulose, and 48.8% lactose, 5% sodium lauryl sulfate, 3% croscarmellose sodium and 1% magnesium stearate. The pharmacokinetic profile of this composition was measured by administering a single 1 mg/kg dose to a panel of 3 fasted beagle dogs and then measuring the amount of Compound A in the blood of the dogs for a period of at least 24 hours. The pharmacokinetic data was as follows: AUC₀₋₂₄ is 52.4±15.9 μM*hr, Cₘₐₓ is 3.46±1.59 μM, and Tₘₐₓ is 4.0 hr with a range of 4.0-24.0 hr.

1. A composition comprising an active pharmaceutical ingredient which is 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile or a pharmaceutically acceptable salt thereof sufficiently mixed in a concentrating enhancing polymer and optionally one or more surfactants, wherein the composition demonstrates a measured transient concentration in excess of any of the crystalline forms of the active pharmaceutical ingredient in any water based media.

2. A pharmaceutical composition comprising an effective amount of an active pharmaceutical ingredient which is 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile or a pharmaceutically acceptable salt thereof and a concentrating enhancing polymer and optionally one or more surfactants, wherein the concentrating enhancing polymer is selected from the group consisting of: hydroxypropyl methyl cellulose acetate succinate, hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone, and polyvinylpyrrolidinone-polyvinylacetate copolymers, wherein the active pharmaceutical ingredient is in substantially amorphous form dispersed in the concentrating enhancing polymer.

3. The pharmaceutical composition according to claim 2, wherein the drug load of the active pharmaceutical ingredient is from about 5% to about 40%.

4. The pharmaceutical composition according to claim 3 wherein the concentrating enhancing polymer is selected from the group consisting of: hydroxypropyl methyl cellulose acetate succinate, hydroxypropyl methyl cellulose phthalate, cellulose acetate phthalate, and cellulose acetate trimellitate.

5. The pharmaceutical composition according to claim 4 wherein the concentrating enhancing polymer is hydroxypropyl methyl cellulose acetate succinate.

6. The pharmaceutical composition of claim 5, wherein said composition comprises one or more surfactants selected from the group consisting of anionic surfactants and nonionic surfactants.

7. The pharmaceutical composition of claim 6, wherein the one or more surfactants is selected from sodium dodecyl sulfate and one or more nonionic surfactants selected from (a) sorbitan fatty acid esters, (b) polyoxyethylene sorbitan fatty acid esters, (c) polyoxyethylene castor oils, (d) polyoxyethylene hydrogenated castor oils, and (e) vitamin E TPGS; and mixtures thereof.

8. The pharmaceutical composition according to claim 1, wherein the active pharmaceutical ingredient is anhydrous 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile.

9. A process for making a composition comprising an active pharmaceutical ingredient which is 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile or a pharmaceutically acceptable salt thereof sufficiently mixed in a concentrating enhancing polymer, comprising
(a) dissolving the active pharmaceutical ingredient and the concentrating enhancing polymer in a solvent system that results in a stable, single-phase dispersion; and
(b) drying the solution from step (a).

10. The process according to claim 9 wherein the drying in step (b) is achieved using a spray drying process comprising:
(i) delivering the solution from step (a) to an atomiser of a spray-drying apparatus;
(ii) dispersing the solution from step (a) into droplets by passing the solution through the atomiser into a drying chamber of the spray-drying apparatus;
(iii) mixing the droplets in the drying chamber with a drying gas which flows at a drying gas flow rate through the drying chamber from an inlet to an outlet of the drying chamber, whereby solvent from the solvent system is evaporated to make particles comprising the concentrating enhancing polymer and active pharmaceutical ingredient, and
(iii) separating the particles from the drying gas and collecting the particles.

11. The process of claim 10 wherein the composition is a pharmaceutical composition and the active pharmaceutical ingredient is present in an effective amount.

12. The process according to claim 10 wherein the particles comprising the concentrating enhancing polymer and active pharmaceutical ingredient resulting from the spray drying process of step (b) is subsequently subjected to a secondary drying process comprising heating the particles and mixing the particles with a second drying gas to evaporate residual solvent from the solvent system, wherein the temperature, relative humidity and/or gas flow rate of the second drying gas are such to ensure that the active pharmaceutical ingredient remains <5% crystalline throughout the duration of the secondary drying process.

13. The process according to claim 10, wherein the spray drying process of step (b) uses a temperature of the drying gas at the inlet of the drying chamber, and ratio of spray solution flow rate to drying gas flow rate to ensure the glass transition temperature of the particles comprising the concentrating enhancing polymer and active pharmaceutical ingredient is greater than about 5° C. over the temperature of the spray dried powder from the moment the particles are collected until the start of the secondary drying process.

14. The process according to claim 10, wherein said spray drying process of step (b) uses a temperature of the drying gas at the inlet of the drying chamber and ratio of spray solution flow rate to drying gas flow rate to ensure the glass transition temperature of the particles comprising the concentrating enhancing polymer and active pharmaceutical ingredient is greater than about 10° C. over the temperature of the spray dried powder from the moment the particles are collected until the start of the secondary drying process.

15. The process according to claim 10, wherein said spray drying process of step (b) uses a temperature of the drying gas at the inlet of the drying chamber and ratio of spray solution flow rate to drying gas flow rate to ensure the glass transition temperature of the particles comprising the concentrating enhancing polymer and active pharmaceutical ingredient is greater than about 20° C. over the temperature of the spray dried powder from the moment the particles are collected until the start of the secondary drying process.

16. The process according to claim 10 wherein prior to delivering the solution from step (a) to the atomiser of a spray-drying apparatus, the solution from step (a) is contacted with a heat exchanger to deliver the solution at an elevated temperature.

17. The process according to claim 10, wherein the pharmaceutical composition is in the form of a tablet, further comprising blending the particles comprising the concentrating enhancing polymer and active pharmaceutical ingredient following the secondary drying process with a diluent, and optionally one or more other excipients, to form a mixture, granulating the mixture and compressing the granulating mixture to form the tablet.

18. The process according to claim 17, wherein the dispersion particles comprising the concentrating enhancing polymer and active pharmaceutical ingredient following the secondary drying process possess a bulk density in the range of 0.1-0.3 g/cc.

19. A process for making a composition comprising an active pharmaceutical ingredient which is 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile or a pharmaceutically acceptable salt thereof sufficiently mixed in a concentrating enhancing polymer, said process comprising dissolving the active pharmaceutical ingredient and the concentrating enhancing polymer in a solvent system and subsequently creating a supersaturated condition so as to precipitate a solid from said solution.

20. The process according to claim 19 further comprising dissolving the active pharmaceutical ingredient and the concentrating enhancing polymer in a solvent with subsequent addition of an anti-solvent, or changing the temperature, so as to precipitate the active pharmaceutical composition and concentrating enhancing polymer from the solution.

21. The process according to claim 10 wherein the spray dried particles of step (b) comprising the concentrating enhancing polymer and active pharmaceutical ingredient have a glass transition temperature that is about 5° C. or more above the storage temperature of the spray dried particles.

22. The process according to claim 10, wherein the spray dried particles of step (b) comprising the concentrating enhancing polymer and active pharmaceutical ingredient have a glass transition temperature that is greater than about 10° C. above the storage temperature of the spray dried particles.

23. The process according to claim 10, wherein the spray dried particles of step (b) comprising the concentrating enhancing polymer and active pharmaceutical ingredient have a glass transition temperature that is about 20° C. or more above the storage temperature of the spray dried particles.

24. The process according to claim 10, wherein the spray dried particles of step (b) comprising the concentrating enhancing polymer and active pharmaceutical ingredient having a glass transition temperature that is greater than about 20° C. above the storage temperature of the spray dried particles.

25. The process according to claim 10 wherein the solvent system is acetone/water.

26. The process according to claim 9, wherein the active pharmaceutical ingredient is 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile.

27. A pharmaceutical composition comprising an effective amount of an active pharmaceutical ingredient which is 3-chloro-5-({1-[(4-methyl-5-oxo-4,5-dihydro-1H-1,2,4-triazol-3-yl)methyl]-2-oxo-4-(trifluoromethyl)-1,2-dihydropyridin-3-yl}oxy)benzonitrile or a pharmaceutically acceptable salt thereof sufficiently mixed in a concentrating enhancing polymer, wherein the pharmaceutical composition is made by the process according to claim 9.
